(19) United States
(12) Patent Application Publication
(10) Pub. No.: US 2018/0291458 A1
(43) Pub. Date: Oct. 11, 2018

(54) USE OF GASTRIC CANCER GENE PANEL

(71) Applicant: NANJING KDRB BIOTECHNOLOGY INC., LIMITED, Jiangsu (CN)

(72) Inventors: Bo HANG, Jiangsu (CN); Pin WANG, Jiangsu (CN); Bin LI, Jiangsu (CN); Jianhua MAO, Jiangsu (CN)

(73) Assignee: NANJING KDRB BIOTECHNOLOGY INC., LIMITED, Jiangsu (CN)

(21) Appl. No.: 15/578,189

(22) PCT Filed: Dec. 22, 2016
(86) PCT No.: PCT/CN2016/111536

§ 371 Date: Nov. 29, 2017

(30) Foreign Application Priority Data
Jun. 15, 2016 (CN) 201610427870.6

(51) Int. Cl. C12Q 1/6886

(52) U.S. Cl. CPC C12Q 1/6886; C12Q 2600/112; C12Q 2600/118; C12Q 2600/106; C12Q 2600/158

(57) ABSTRACT

Disclosed is use of a panel of gastric cancer (GC)-related genes in clinical applications. The present invention is based on a panel of 53 genes related to prognosis in GC and detection of their expression levels in clinical samples to calculate prognostic scores, so as to evaluate clinical prognosis of GC patients and its other applications. This score system is useful for assisting in treatment selection for GC patients and predicting the response to therapeutic intervention, to determine the degree of benefit of patients from chemotherapy and targeted therapy, thus avoiding overtreatment, reducing medical cost, and achieving personalized medicine. Accordingly, a 53-gene expression assay kit is designed and developed according to this system and different detection technology platforms.

### RELATED APPLICATIONS

This application is a U.S. National Phase of and claims priority to International Patent Application No. PCT/CN2016/111536, International Filing Date Dec. 22, 2016, which claims benefit of Chinese Patent Application No. 201610427870.6 filed Jun. 15, 2016; both of which are hereby expressly incorporated by reference in their entireties for all purposes.

### BACKGROUND OF THE INVENTION

### Field of the Invention

The present invention relates to the field of biomarkers and therapeutic targets, and more particularly, to use of a panel of gastric cancer related genes in clinical applications.

### Description of Related Art

Gastric cancer (GC) is a malignant tumor initiated from the epithelial cells of gastric mucosa. GC has been one of the most common malignant tumors in the world and ranks fifth in the incidence rate, following lung cancer, breast cancer, colorectal cancer, and prostate cancer. Despite of the slightly reduced overall incidence and mortality of GC over the past decade, to date, the incidence and mortality of GC still remains very high. Moreover, the number of people suffering from GC follows an upward trend, and there are about one million of new cases each year. About 400 thousand new cases occur annually in China, accounting for 42% of all cases worldwide. From data published on the official site of National Health and Family Planning Commission of the People's Republic of China (NHFPC), GC morbidity rates of rural and urban residents are 18.12/100 thousand and 19.05/100 thousand respectively on 2005, 19.66/100 thousand and 22.09/100 thousand on 2006, 22.87/100 thousand and 23.35/100 thousand on 2007, 18.60/100 thousand and 26.33/100 thousand on 2008, 18.17/100 thousand and 23.10/100 thousand on 2009, 18.63/100 thousand and 22.57/100 thousand on 2010, and 19.66/100 thousand and 22.09/100 thousand on 2011. The GC studies in China have indicated that GC is one of the top three ranks in morbidity and mortality rates of malignant tumors, and GC is still a main focus in prevention and treatment of tumors in China.

With the advances in science and biotechnology, the level of early diagnosis for GC has been improved to certain extent, which, in turn, significantly improves its five-year survival rate. Even so, the five-year survival rate of advanced GC is only about 29.3%, mainly because GC is not easily diagnosed at an early stage and is discovered lately, so that the best treatment time is missed, and recurrence and metastasis of GC may easily occur. The treatment of GC is divided primarily into surgery, radiotherapy, chemotherapy, targeted therapy, etc. Chemotherapy is an important treatment regimen for patients with advanced/metastatic GC, commonly associated with serious side effects. Recently, targeting agents representative of trastuzumab open new ways for the targeted therapy of GC. Currently, trastuzumab in combination with chemotherapy has become a first choice for patients for which human epidermal growth factor receptor 2 (HER2/ERBB2) gene amplification or over-expression is positive.

GC is a polygenic disease, where the interactions of various cancer genes with the microenvironment in vivo lead to the early lesions of gastric mucosa to the dysplasia, and ultimately to the development of GC. The characteristically differential expression of related genes can be observed throughout the whole process. In clinical practice, there has been a lack of corresponding molecular markers for the distinguishment of GC staging and degree of differentiation. Recently, there is increasing evidence that the molecular characteristics of GC tissues also play an important role in the prognosis. For example, about 10-30% of GC patients have amplification or over-expression of HER2/ERBB2 gene, and the later is closely associated with the prognosis and lymph node metastasis of GC. Also, evidence suggests that the accumulation of p53 protein is negatively correlated with the prognosis of GC. In addition, the transcription factor hypoxia-inducible factor 1α (HIF-1α) is highly expressed in GC cells, and exhibits an even higher expression in patients with GC at the early stage as identified by TNM classification, which may be related to early development of GC.

In the current cancer research, the chip technology and the next-generation sequencing technology have become important tools for investigating genetic heterogeneity and complexity of somatic cells in GC, and provide enormous amounts of information for development of biomarkers related to diagnosis, treatment and prognosis. Gene expression profiling can classify the same tumor into different subtypes and enable the investigation of their prognosis. The construction of a gene correlation network using gene expression profiling technology proves to be critical for the understanding of cancer initiation and development. For example, a GC regulatory network is constructed with CDKNIA as the node, and seven genes related to GC occurrence (i.e., MMP7, SPARC, SOD2, INHBA, IGFBP7, NEK6, and LUM) are identified. The results show that these seven genes are activated as the disease progresses, indicating that these genes may be associated with cancer development.

As to other tumors, the gene testing techniques, Oncotype DX developed by Genomic Health Inc. in United States and MammaPrint developed by Agendia Inc. in Norway, can be used to evaluate the prognosis for recurrence and metastasis of breast cancer, and provide instructional information about whether patients needs to be treated with chemotherapy. Oncotype DX is a quantitative reverse transcriptase polymerase chain reaction (RT-PCR)-based test measuring expression of 21 genes on RNA from tissue specimens in ER-positive, lymph node-negative breast cancer, including 16 recurrence-related target genes (proliferation, invasion, HER2, hormones) and 5 reference genes. Patients with breast cancer are categorized into low-risk (RS<18), intermediate-risk (RS 18 to 30), and high-risk (RS≥31) groups in terms of 10-year risk of recurrence, to determine whether patients need to be treated with chemotherapy. Generally, chemotherapy is not recommended for patients with low RS, and is recommended for patients with high RS. For intermediate RS, a recommendation on whether or not to carry out chemotherapy is primarily dependent on age and health of patients. MammaPrint serves to predict recurrence in patients with ER positive and ER negative plus lymph node-negative breast cancer using the expression of 70 genes, and is superior to clinicopathological indexes in predicting metastasis and survival. Both tests have been approved for marketing by the FDA in the United States. In addition, Oncotype DX has been listed as a test item of breast cancer in the NCCN Guidelines and in U.S. Health Insurance. Although genetics and genomics are related to each other, both provide different types of information. A genetic test generally serves to screen for genetic risk factors with which a disease or cancer may develop, while a genomic test, such as Oncotype DX, serves to evaluate the activity of a panel of important cancer-related genes to disclose biological properties of a tumor in a particular individual and more accurately predict the behavior of the tumor.

Genomic Health Inc. also has developed an Oncotype DX gene test item for prostate cancer and colon cancer. However, to date, no similar test has been reported for the prognosis of GC in the world. It is accordingly highly necessary to design and develop a multi-gene expression profiling and prognostic scoring system for GC on the basis of the prior art knowledge and techniques.

### SUMMARY OF THE INVENTION

### Technical Problem to be Solved

The present invention comprehensively identifies 249 related cancer biomarkers by establishing a multi-step meta-analytic approach using publically available international tumor datasets; and then identifies the key genes related to the prognosis of GC by stepwise multivariate clustering techniques. Based on these analyses, we created a 53-gene expression profiling and prognostic scoring system and successfully applied it to predict the survival in the clinical data of GC. This method is useful for assisting in treatment selection of GC patients and predicting the response to therapeutic intervention, to determine the degree of benefit of patients from chemotherapy/targeted therapy, thus avoiding overtreatment and reducing medical cost.

### Technical Solution

To achieve the foregoing objective, the present invention adopts the following technical solution:

A multi-gene expression profiling and prognostic scoring system for evaluating the prognosis of GC. The present invention includes 53 genes related to the prognosis of GC and detection of their expression levels in clinical samples, and then prediction of clinical prognosis by calculating prognostic scores.

Preferably, firstly, we identified genes significantly differentially expressed in GC by a comparison between normal and GC tissues. We developed a multi-step strategy to identify a critical gene signature that is able to distinguish good and bad prognosis for GC patients. We used two publically available international tumor datasets: (1) the Cancer Genome Atlas (TCGA) generated by RNA sequencing; and (2) human gastric tumor and normal tissue banks GSE30727 generated by Affymetrix chip (Affymetrix Genechip arrays, HG-U133 Plus 2.0). We found that 688 and 3239 genes reached our selection criteria (2 fold changes in expression and adjusted p-value <0.05) in TCGA and GSE30727, respectively. 276 genes were found to be overlapping between TCGA and GSE30727 datasets, including 57 genes downregulated and 219 genes unregulated in GC.

Preferably, we further assessed the importance of differential expression of the above 276 genes in clinical development of GC. We evaluated their prognostic value for GC patients in a large public clinical chip GC dataset using an on-line tool for the prognosis of survival, Kaplan-Meier plotter (http://kmplot.com/analysis/index.php?p=service&cancer=gastric). These genes were divided into two groups (high and low expression) based on their expression levels. Subsequently, the effects of high or low expression level of these genes on the 5-year survival of GC patients were assessed using the Kaplan-Meier curves (FIG. 1), where 249 genes were found to be significantly associated with overall survival. This result suggested that these molecular markers may provide an effective prediction for the treatment prognosis of GC patients. Finally we ranked the importance of the genes on clinical prognosis according to their p-values derived from univariate analysis (Table 1), as the criteria for the subsequent choice of genes.

Preferably, we created a gene co-expression network of 249 genes in GC, in order to better reveal the biological functions of these genes and the molecular mechanism underlying GC development. Using the Database for Annotation, Visualization and Integrated Discovery (DAVID), we observed that these genes are significantly enriched for regulating cell proliferation, adhesion and migration, RNA/ncRNA process, acetylation, extracellular matrix organization, etc. (FIG. 2), all of which are hallmarks of cancer. Next, we constructed a co-expression network (FIG. 2) of genes related to the biological functions based on the correlation network analysis software (http://baderlab.org/Software/ExpressionCorrelation) using TCGA data.

Preferably, we developed a prognostic scoring system for GC based on the above results. We applied a stepwise canonical discriminant analysis to identify a gene signature that is able to classify patients into good or bad prognosis with 100% accuracy. Finally we identified 53 specific biomarker genes for the prognosis of GC, and the scoring system yielded 100% accuracy in prognosis prediction. The genes specifically include: (1) cell cycle related genes: CEP55, MCM2, PRC1, SCNN1B, TUBB; (2) acetylation related genes: ADNP, ABCE1, CBFB, CHORDC1, CCT6A, GART, SMS; (3) RNA/ncRNA process related genes: NOL8, NCL, PN01; (4) extracellular matrix related genes: APOE, APOC1, CXCL10, COL6A3, CPXM1, GABBR1, INHBA, LAMC2, MMP14, TNFAIP2; and (5) other genes: ADH1C, ALDH6A1, ATP13A3, BAZ1A, BCAR3, CAPRIN1, CXCL1, CCT2, ECHD2, ETFDH, ENC1, EPHB4, FHOD1, FGFR4, KAT2A, KLF4, LRRC41, LIMK1, OSMA, PTGS1, PGRMC2, P4HA1, PDP1, PRR7, SCC12A9, SLC20A1, TGS1, and TCERG1 (FIG. 3).

The prognostic scoring system predicts survival probability of a GC patient using the calculated prognostic score. A prognostic score was defined as the linear combination of gene expression levels based on canonical discriminant function. The calculation formula is shown below:

Prognostic score:=Σᵢ₌₁⁵³(Canonical discriminant function coefficient)*(gene expression level)

Note: The canonical discriminant function coefficients are presented in Table 2.

If the prognostic score is ≤−2, we defined that the patient had good signature; and if the prognostic score is >−2, we defined the patient as bad signature (Refer to FIG. 4). We evaluated the accuracy of this prognostic scoring system using data from the TCGA dataset. As shown in FIG. 5, the patients with good signature had significantly longer survival than those with bad signature. More than 50% of patients with good signature still survived after 100 months while all patients with bad signature died before 80 months. In conclusion, our test results had shown the distributions of prognostic score in good and bad prognostic patients were clearly discriminative (FIG. 5), indicating that this prognostic scoring system has its discriminative ability to distinguish good prognostic patients from bad prognostic patients. We obtained similar accurate results using the GSE15459 dataset (Refer to Example 2 and FIG. 6).

Preferably, we accordingly designed and developed an assay kit and a scoring system, by collecting RNA of tumor tissues of patients with GC, including but not limited to, fresh biopsy tissue, post operative tissue, fixed tissue, and paraffin-embedded tissue, according to different detection technology platforms, including but not limited to real-time, fluorescence-based quantitative PCR, gene chip, second-generation high-throughput sequencing, Panomics, and Nanostring technologies. The kit developed by the present invention designs respective gene primers (real-time, fluorescence-based quantitative PCR) and target probes (gene chip, next-generation sequencing, Panomics, and Nanostring technologies) for different technology platforms.

Prognostic score defined in this invention (≤−2 and >−2) is made according to data from TCGA dataset based on next-generation sequencing. The absolute value and cutoff score of prognostic score can vary depending on different detection technology platforms, and need to be adjusted respectively.

### Advantageous Effect

Although some researches of molecular characteristics have been carried out in GC, it has been rarely reported that researches attempt to find gene signature associated with the prognosis of GC, and it has not yet been reported that a prognostic scoring system is applied clinically. The present invention successfully found a panel of 53 important biomarker genes for predicting overall survival of GC patients using multi-omics data, and for the first time established a prognostic scoring system based on a 53-gene signature. We also showed that the prognostic scores of the system are able to distinguish patients with good prognosis from those with bad prognosis. This invention is useful for assisting in treatment selection of GC patients and predicting the response to therapeutic intervention, to determine the degree of benefit of patients from chemotherapy and targeted therapy, thus avoiding overtreatment, reducing medical cost, and achieving personalized medicine.

### BRIEF DESCRIPTION OF THE DRAWINGS

FIG. 1 shows examples of Kaplan-Meier survival curves for GC related genes. P values are obtained by log-rank test that compares between two groups.

FIG. 2 shows a co-expression network diagram for the GC genes according to this invention.

FIG. 3 shows 53 genes in the prognostic scoring system for GC and related functions/pathways according to this invention.

FIG. 4 shows a distribution of prognostic score in good and bad GC prognostic patients according to this invention.

FIG. 5 shows that Kaplan-Meier survival curves indicating that prognostic score is significantly correlated with overall survival for GC in TCGA dataset.

FIG. 6 shows that Kaplan-Meier survival curves indicating that prognostic score is significantly correlated with overall survival for GC in GSE15459 dataset.

FIG. 7 shows that overall survival of patients cannot be predicted based on analysis of the reported 19- and 7-gene signatures (TCGA data).

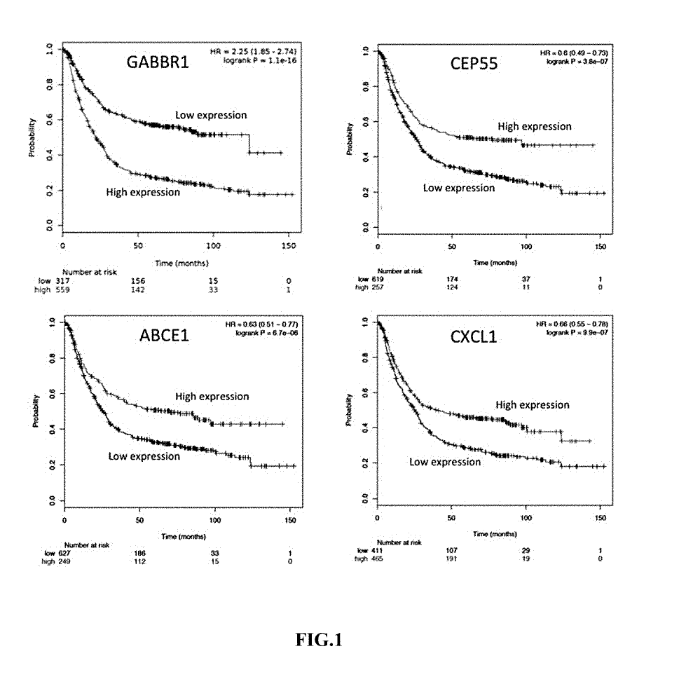

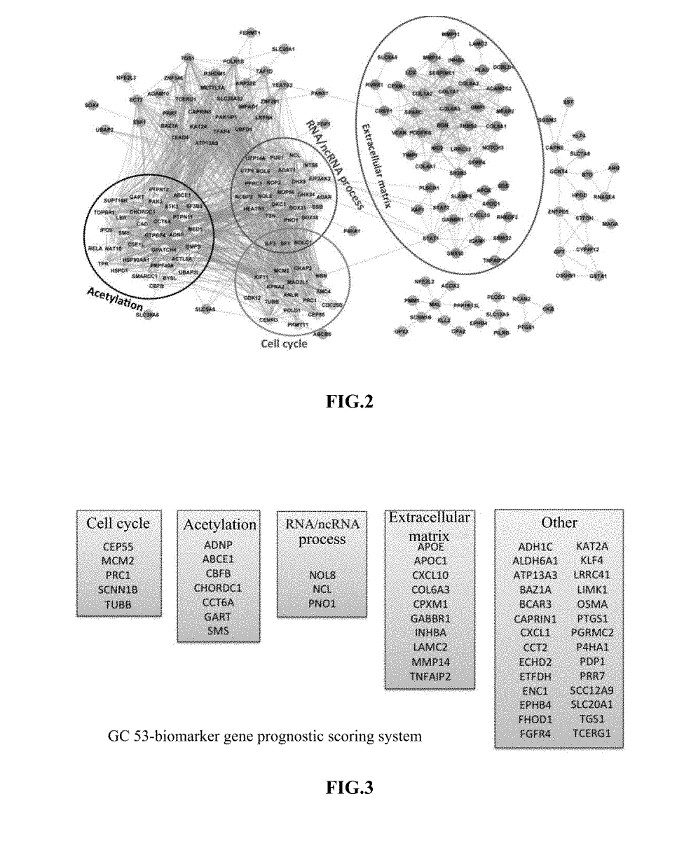

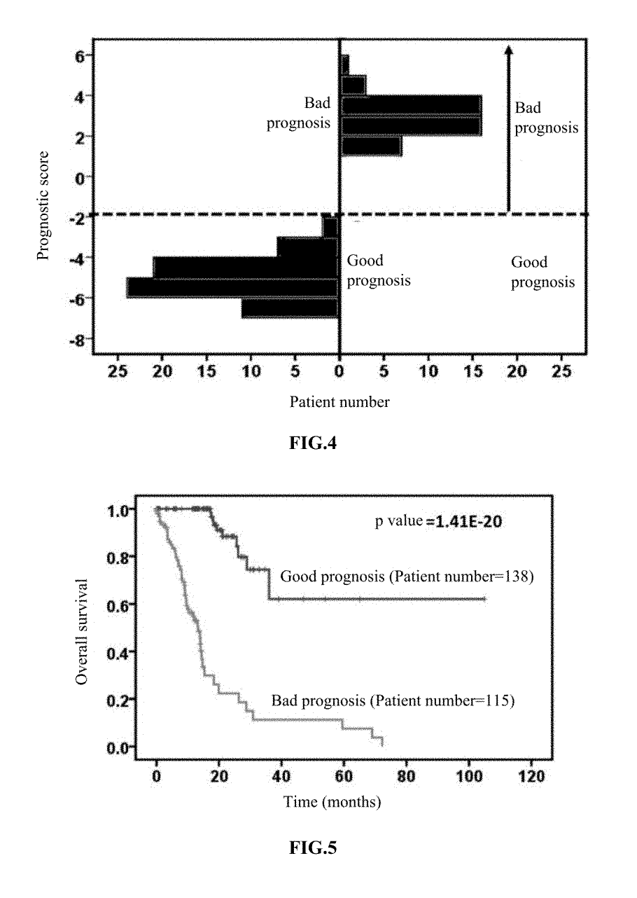

### DETAILED DESCRIPTION OF THE INVENTION

The invention will be set forth further below with reference to the accompanying drawings and specific embodiments. It should be understood that these embodiments are merely used to illustrate the present invention and are not intended to limit the scope of the present invention. Various equivalent modifications of the present invention made by those skilled in the art after reading the present invention, all fall within the scope defined by the appended claims of the present application

### Example 1

Validation of the System Using GC Patients in the TCGA Public Dataset:

The prognostic scoring system was applied to 253 GC patients in TCGA having survival data. Prognostic score was used to predict survival probability for each individual patient. We divided patients into two groups based on prognostic score. If the prognostic score is ≤−2, we defined that the patient had good signature; and if the prognostic score is >−2, we defined the patient as bad signature. As shown in FIG. 5, the patients with good signature had significantly longer survival than those with bad signature. More than 50% of patients with good signature still survived after 100 months while all patients with bad signature died before 80 months.

Some documents used differential expression to show correlation of a gene or multigene panel with the prognosis of GC. One question was whether our 53-gene scoring system was better than the above monogenic or genomic system. We first carried out univariate Cox regression analysis, indicating that any single gene from the above-mentioned 276 genes from TCGA was generally weakly associated with overall survival for GC. Then, we used previously reported gene signatures for GC to calculate prognostic score, including a 19-gene panel (Cui J et al., Gene-Expression Signatures Can Distinguish Gastric Cancer Grades and Stages. PLoSONE. 2011; 6: E17819) and 7-gene signature (TakenoA et al., Integrative approach for differentially over-expressed genes in gastric cancer by combining large-scale gene expression profiling and network analysis. British. J. Cancer. 2008; 99: 1307-15). As shown in FIG. 7, the scoring analysis of both multi-gene panel signatures could not clearly predict overall survival of patients in TCGA data.

### Example 2

Survival Validation Using GC Patients in the GSE15459 Public Dataset:

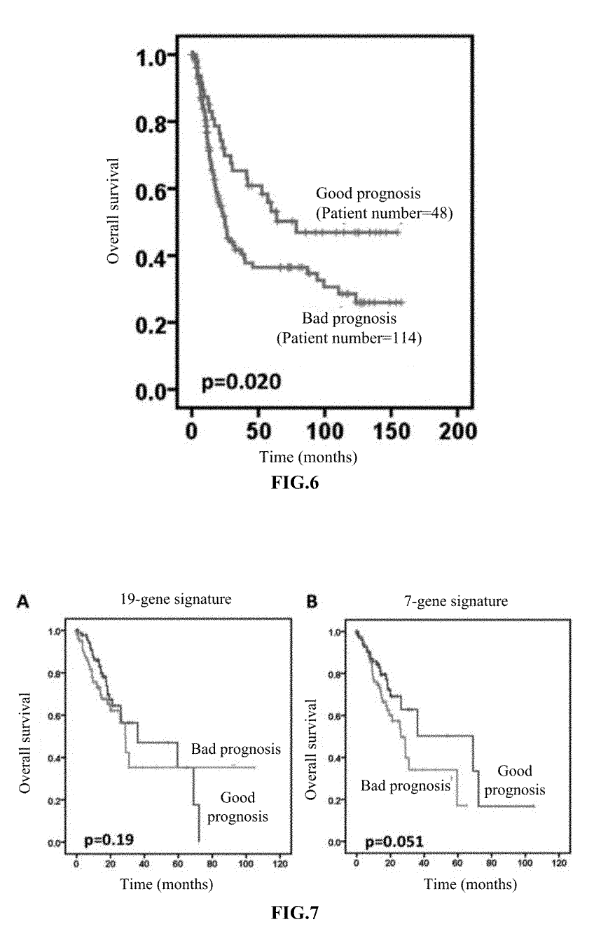

Using the same method, we validated the application value of the prognostic scoring system in the GSE15459 public dataset. Although gene expression values of GC tissues in this dataset were determined by Affymetrix chip technology, causing the difference in expression level baseline and scale and thus the difference in absolute value of prognostic score, the scoring system of this invention can still successfully predict the prognosis of GC (FIG. 6).

### Example 3

Prediction for Prognosis in Clinical GC Patients:

Tumor tissues of GC patients received clinically were collected and RNA was extracted. The tumor tissues could include fresh biopsy tissue, post operative tissue, fixed tissue, and paraffin-embedded tissue. Then, the expression levels of 53 genes in the prognostic scoring system were quantitatively determined using the kit developed by this invention and the corresponding apparatus. The expression levels of 53 genes were input into the prognostic scoring formula established by this invention:

Prognostic score:=Σᵢ₌₁⁵³(Canonical discriminant function coefficient)*(gene expression level)

After the prognostic score of patients was calculated, the prognosis of patients, for example, 5-year survival, was predicted by the physicians according to the score values (Refer to Example 1). Currently, we established a model by retrospective study, and successfully validated this prognostic scoring system in different datasets. Also, prospective study was initiated to further improve the scoring system.

### Example 4

Prediction of Response of Clinical GC Patients to HER2/ERBB2 Targeted Therapy (Such as but not Limited to Lapatinib and Trastuzumab):

About 10-30% of GCs had amplification or over-expression of HER2/ERBB2, as prognosis and prediction biomarkers. Currently, only part of GC patients with positive HER2/ERBB2 are responsive to HER2 targeted therapy. In order to reduce ineffective or excessive use of the targeting agent and reduce the medical cost, the present invention predicted response of clinical GC patients to HER2/ERBB2 targeted agent (such as but not limited to Lapatinib and Trastuzumab) as follows:

Tumor tissues of GC patients received clinically and with positive HER2/ERBB2 were collected and RNA was extracted. The tumor tissues could include fresh biopsy tissue, post operative tissue, fixed tissue, and paraffin-embedded tissue. Then, the expression levels of 53 genes in the prognostic scoring system were quantitatively determined using the kit developed by this invention and the corresponding apparatus. Next, the expression levels of 53 genes were input into the prognostic scoring formula established by this invention:

Prognostic score:=Σᵢ₌₁⁵³(Canonical discriminant function coefficient)*(gene expression level)

After the prognostic score of patients is calculated, whether to receive the HER2/ERBB2 targeted therapy will be considered by the physicians according to the score values. For patients marked with good prognosis in prognostic score, it is recommended for the physicians to appropriately consider the necessity of HER2/ERBB2 targeted therapy, thus avoiding overtreatment, reducing medical cost, and achieving personalized medicine.

### Example 5

Prediction of Response of Clinical GC Patients to Chemotherapeutic Agent 5-FU:

Currently, the total response rate of chemotherapy for GC is about 30%. In order to reduce ineffective or excessive dosing and reduce the medical cost, the present invention predicted response of clinical GC patients to chemotherapeutic agent 5-FU:

Tumor tissues of GC patients received clinically were collected and RNA was extracted. The tumor tissues could include fresh biopsy tissue, post operative tissue, fixed tissue, and paraffin-embedded tissue. Then, the expression levels of 53 genes were quantitatively determined using the kit developed by this invention and the corresponding apparatus. The expression levels of 53 genes were input into the prognostic scoring formula established by this invention:

Prognostic score:=Σᵢ₌₁⁵³(Canonical discriminant function coefficient)*(gene expression level)

After the prognostic score of patients is calculated, whether to receive the 5-FU chemotherapy will be considered by the physicians according to the score values. For patients marked with good prognosis in prognostic score, it is recommended for the physicians to appropriately consider the necessity of 5-FU chemotherapy. For patients marked with bad prognosis in prognostic score, it is recommended for the physicians to appropriately consider the increase in treatment intensity of 5-FU or other chemotherapeutic agents.

TABLE 1
K-M Summary of results from K-M plotter analysis.
(If a gene has multiple Affymetrix probes, the most
significant one is listed in this table.)

| Gene Name | Rank | Hazard ratio (HR) | 95% CI | p value |
|---|---|---|---|---|
| NOTCH3 | 1 | 2.83 | 2.22-3.59 | <1.0E−16 |
| SPRY4 | 2 | 2.34 | 1.91-2.88 | <1.0E−16 |
| TMEM63A | 3 | 2.3 | 1.88-2.8 | <1.0E−16 |
| R3HDM1 | 4 | 2.2 | 1.82-2.66 | <1.0E−16 |
| UBAP2L | 5 | 2.31 | 1.88-2.84 | 1.1E−16 |
| GABBR1 | 6 | 2.25 | 1.85-2.74 | 1.1E−16 |
| RAD23A | 7 | 2.29 | 1.87-2.81 | 2.2E−16 |
| GPX3 | 8 | 2.57 | 2.03-3.26 | 4.4E−16 |
| FHOD1 | 9 | 2.03 | 1.71-2.42 | 4.4E−16 |
| KAT2A | 10 | 0.62 | 0.56-0.7 | 6.7E−16 |
| SF1 | 11 | 2.15 | 1.78-2.61 | 7.8E−16 |
| PTPN12 | 12 | 2.38 | 1.91-2.97 | 1.7E−15 |
| RUNX1 | 13 | 2.25 | 1.83-2.76 | 1.8E−15 |
| SOX4 | 14 | 2.05 | 1.71-2.46 | 3.1E−15 |
| ILF3 | 15 | 2.29 | 1.85-2.84 | 3.7E−15 |
| BMP1 | 16 | 2.17 | 1.78-2.65 | 4.7E−15 |
| SMARCC1 | 17 | 0.45 | 0.36-0.55 | 4.9E−15 |
| DKC1 | 18 | 1.57 | 1.4-1.76 | 1.2E−14 |
| LY6E | 19 | 2.08 | 1.72-2.52 | 1.3E−14 |
| PILRB | 20 | 1.94 | 1.63-2.3 | 1.8E−14 |
| GPATCH4 | 21 | 2.07 | 1.71-2.51 | 3.7E−14 |
| COL1A1 | 22 | 2.33 | 1.86-2.92 | 5.0E−14 |
| COL4A1 | 23 | 2.33 | 1.86-2.92 | 5.0E−14 |
| PVR | 24 | 1.98 | 1.65-2.37 | 5.2E−14 |
| TUBB | 25 | 1.89 | 1.59-2.24 | 1.1E−13 |
| PAK2 | 26 | 2.02 | 1.66-2.45 | 4.9E−13 |
| SBNO2 | 27 | 2.01 | 1.66-2.45 | 7.9E−13 |
| OSGIN1 | 28 | 1.84 | 1.55-2.18 | 9.5E−13 |
| DRG2 | 29 | 1.94 | 1.61-2.34 | 1.2E−12 |
| CBFB | 30 | 1.86 | 1.56-2.22 | 2.0E−12 |
| RA114 | 31 | 1.82 | 1.53-2.15 | 2.9E−12 |
| SNX10 | 32 | 0.49 | 0.4-0.6 | 3.2E−12 |
| CSNK1D | 33 | 1.84 | 1.55-2.2 | 4.3E−12 |
| BCL2A1 | 34 | 0.49 | 0.4-0.61 | 5.3E−12 |
| NOP2 | 35 | 1.82 | 1.53-2.17 | 5.5E−12 |
| IPO9 | 36 | 1.8 | 1.52-2.13 | 7.2E−12 |
| ADAMTS2 | 37 | 1.81 | 1.53-2.16 | 7.9E−12 |
| PDGFRB | 38 | 2.17 | 1.72-2.72 | 9.7E−12 |
| ALDOC | 39 | 1.79 | 1.51-2.12 | 1.1E−11 |
| STK3 | 40 | 1.79 | 1.51-2.13 | 1.2E−11 |
| ZNF281 | 41 | 0.55 | 0.46-0.65 | 1.7E−11 |
| TNFAIP2 | 42 | 1.77 | 1.49-2.1 | 3.1E−11 |
| ABCB8 | 43 | 1.77 | 1.49-2.11 | 3.9E−11 |
| NBN | 44 | 0.48 | 0.39-0.6 | 4.9E−11 |
| POLR1B | 45 | 1.79 | 1.5-2.14 | 7.3E−11 |
| NFE2L2 | 46 | 0.57 | 0.48-0.68 | 1.1E−10 |
| BGN | 47 | 1.9 | 1.56-2.32 | 1.3E−10 |
| DHX9 | 48 | 0.51 | 0.41-0.63 | 1.4E−10 |
| DDX18 | 49 | 0.5 | 0.4-0.62 | 1.9E−10 |
| TIMP1 | 50 | 1.92 | 1.57-2.36 | 2.2E−10 |
| ADAR | 51 | 1.82 | 1.51-2.19 | 2.4E−10 |
| TRRAP | 52 | 1.88 | 1.54-2.29 | 2.6E−10 |
| SMS | 53 | 0.58 | 0.49-0.69 | 3.6E−10 |
| SLC12A7 | 54 | 1.74 | 1.46-2.08 | 4.0E−10 |
| PLSCR1 | 55 | 0.57 | 0.48-0.68 | 4.6E−10 |
| ME1 | 56 | 1.81 | 1.49-2.18 | 6.9E−10 |
| CAPRIN1 | 57 | 0.59 | 0.49-0.7 | 8.4E−10 |
| TAF1D | 58 | 1.72 | 1.44-2.05 | 1.0E−09 |
| PTPN11 | 59 | 1.37 | 1.24-1.52 | 1.7E−09 |
| MMP11 | 60 | 1.82 | 1.49-2.23 | 2.0E−09 |
| CTSB | 61 | 1.41 | 1.26-1.59 | 2.4E−09 |
| ECT2 | 62 | 0.52 | 0.42-0.65 | 2.5E−09 |
| PTGS1 | 63 | 1.77 | 1.46-2.15 | 3.3E−09 |
| MAD2L1 | 64 | 0.58 | 0.48-0.7 | 3.3E−09 |
| TEAD4 | 65 | 1.67 | 1.41-1.99 | 4.1E−09 |
| RHBDF2 | 66 | 1.79 | 1.47-2.18 | 4.3E−09 |
| ECHDC2 | 67 | 1.77 | 1.46-2.15 | 4.8E−09 |
| SMC4 | 68 | 1.71 | 1.43-2.06 | 6.0E−09 |
| TFAP4 | 69 | 1.71 | 1.42-2.05 | 6.6E−09 |
| TPR | 70 | 1.66 | 1.39-1.97 | 8.8E−09 |
| ENTPD5 | 71 | 1.65 | 1.38-1.96 | 1.5E−08 |
| SSB | 72 | 0.54 | 0.44-0.67 | 1.7E−08 |
| CKB | 73 | 1.67 | 1.4-2.01 | 1.7E−08 |
| CAD | 74 | 1.62 | 1.37-1.92 | 2.0E−08 |
| GART | 75 | 1.77 | 1.44-2.16 | 2.3E−08 |
| SLC5A6 | 76 | 1.65 | 1.38-1.96 | 2.4E−08 |
| UTP14A | 77 | 1.63 | 1.37-1.95 | 2.4E−08 |
| FGFR4 | 78 | 1.63 | 1.37-1.94 | 2.5E−08 |
| MED1 | 79 | 1.66 | 1.38-1.99 | 2.9E−08 |
| ACTL6A | 80 | 0.58 | 0.48-0.71 | 3.1E−08 |
| METTL7A | 81 | 1.71 | 1.41-2.07 | 3.3E−08 |
| TSN | 82 | 0.56 | 0.46-0.69 | 3.4E−08 |
| HERPUD2 | 83 | 1.84 | 1.48-2.29 | 3.5E−08 |
| PRPF40A | 84 | 1.63 | 1.37-1.94 | 3.6E−08 |
| CYP4F12 | 85 | 1.67 | 1.39-2.01 | 4.2E−08 |
| KPNA2 | 86 | 0.54 | 0.44-0.68 | 4.2E−08 |
| PPPIR13L | 87 | 1.6 | 1.35-1.9 | 5.9E−08 |
| HSPD1 | 88 | 0.59 | 0.49-0.72 | 6.9E−08 |
| UBL3 | 89 | 0.61 | 0.51-0.73 | 7.0E−08 |
| MFAP2 | 90 | 1.59 | 1.34-1.88 | 8.0E−08 |
| COL8A1 | 91 | 1.59 | 1.34-1.88 | 8.5E−08 |
| BAZ1A | 92 | 0.63 | 0.53-0.75 | 9.4E−08 |
| DCBLD1 | 93 | 1.79 | 1.44-2.22 | 9.8E−08 |
| STAT1 | 94 | 0.55 | 0.45-0.69 | 9.0E−08 |
| FAM134A | 95 | 1.58 | 1.33-1.87 | 1.0E−07 |
| CXCL10 | 96 | 0.58 | 0.47-0.71 | 1.0E−07 |
| DDX21 | 97 | 0.53 | 0.42-0.68 | 1.2E−07 |
| ADAM10 | 98 | 0.63 | 0.53-0.75 | 1.3E−07 |
| NFE2L3 | 99 | 0.62 | 0.52-0.74 | 1.3E−07 |
| COL1A2 | 100 | 1.57 | 1.33-1.86 | 1.4E−07 |
| LRRC32 | 101 | 1.68 | 1.38-2.04 | 1.4E−07 |
| ETFDH | 102 | 0.6 | 0.49-0.73 | 1.5E−07 |
| UBFD1 | 103 | 1.62 | 1.35-1.95 | 1.5E−07 |
| ALAD | 104 | 1.57 | 1.32-1.86 | 1.7E−07 |
| CKAP2 | 105 | 0.63 | 0.53-0.75 | 1.9E−07 |
| SLC7A8 | 106 | 1.71 | 1.39-2.09 | 2.1E−07 |
| ESF1 | 107 | 1.35 | 1.21-1.52 | 2.2E−07 |
| SLC12A9 | 108 | 1.87 | 1.47-2.39 | 2.4E−07 |
| RELA | 109 | 1.66 | 1.37-2.02 | 2.6E−07 |
| GSTA1 | 110 | 1.75 | 1.41-2.17 | 2.9E−07 |
| CEP55 | 111 | 0.6 | 0.49-0.73 | 3.8E−07 |
| LRRC41 | 112 | 1.77 | 1.41-2.21 | 3.9E−07 |
| ELL2 | 113 | 1.58 | 1.32-1.88 | 4.0E−07 |
| KIF11 | 114 | 0.58 | 0.46-0.72 | 4.1E−07 |
| SH2B3 | 115 | 1.63 | 1.35-1.98 | 4.8E−07 |
| NAT10 | 116 | 1.55 | 1.31-1.85 | 4.9E−07 |
| PGRMC2 | 117 | 0.65 | 0.55-0.77 | 5.1E−07 |
| CDC25B | 118 | 1.55 | 1.31-1.85 | 5.3E−07 |
| PKMYT1 | 119 | 1.57 | 1.31-1.88 | 7.3E−07 |
| TGS1 | 120 | 0.56 | 0.45-0.71 | 9.1E−07 |
| VCAN | 121 | 1.64 | 1.34-2.01 | 9.4E−07 |
| NOL6 | 122 | 1.58 | 1.31-1.9 | 9.6E−07 |
| PANX1 | 123 | 0.62 | 0.51-0.75 | 9.8E−07 |
| SLC6A6 | 124 | 1.68 | 1.36-2.07 | 9.9E−07 |
| CXCL1 | 125 | 0.66 | 0.55-0.78 | 9.9E−07 |
| PLOD3 | 126 | 1.53 | 1.28-1.81 | 1.3E−06 |
| THBS2 | 127 | 1.55 | 1.29-1.85 | 1.4E−06 |
| LIMK1 | 128 | 1.53 | 1.29-1.83 | 1.5E−06 |
| GNS | 129 | 0.66 | 0.56-0.79 | 2.4E−06 |
| LAMC2 | 130 | 1.5 | 1.27-1.78 | 2.6E−06 |
| PLAU | 131 | 1.5 | 1.26-1.78 | 2.7E−06 |
| LIF | 132 | 1.5 | 1.26-1.77 | 2.8E−06 |
| HSP90AA1 | 133 | 0.63 | 0.51-0.76 | 2.9e−06 |
| PPRC1 | 134 | 1.54 | 1.28-1.85 | 3.3E−06 |
| PUS1 | 135 | 1.5 | 1.26-1.78 | 3.5E−06 |
| ENC1 | 136 | 1.49 | 1.26-1.77 | 3.6E−06 |
| ADNP | 137 | 0.67 | 0.57-0.8 | 3.7E−06 |
| RNASE4 | 138 | 0.65 | 0.54-0.78 | 4.3E−06 |
| SF3B3 | 139 | 1.55 | 1.28-1.87 | 4.6E−06 |
| ABCE1 | 140 | 0.63 | 0.51-0.77 | 6.7E−06 |
| CHORDC1 | 141 | 0.62 | 0.5-0.77 | 6.8E−06 |
| ANLN | 142 | 0.6 | 0.47-0.75 | 7.0E−06 |
| LRFN4 | 143 | 1.48 | 1.25-1.76 | 7.4E−06 |
| AMT | 144 | 1.6 | 1.3-1.98 | 7.7E−06 |
| NOLC1 | 145 | 1.47 | 1.24-1.75 | 8.0E−06 |
| P4HA1 | 146 | 0.68 | 0.57-0.81 | 9.0E−06 |
| TCAM1 | 147 | 1.51 | 1.26-1.81 | 9.6E−06 |
| CENPO | 148 | 1.43 | 1.22-1.69 | 1.1E−05 |
| UBAP2 | 149 | 1.5 | 1.25-1.8 | 1.2E−05 |
| DHX34 | 150 | 1.57 | 1.28-1.92 | 1.3E−05 |
| YEATS2 | 151 | 1.48 | 1.24-1.77 | 1.6E−05 |
| ANP32E | 152 | 0.69 | 0.58-0.82 | 1.6E−05 |
| BYSL | 153 | 1.45 | 1.22-1.72 | 2.0E−05 |
| APOE | 154 | 1.45 | 1.22-1.73 | 2.2E−05 |
| CSE1L | 155 | 1.45 | 1.22-1.73 | 2.3E−05 |
| SERPINE1 | 156 | 1.44 | 1.22-1.71 | 2.4E−05 |
| ADAM8 | 157 | 1.44 | 1.21-1.7 | 2.4E−05 |
| SFRP4 | 158 | 1.48 | 1.23-1.77 | 2.4E−05 |
| INHBA | 159 | 1.51 | 1.25-1.84 | 2.5E−05 |
| PMEPA1 | 160 | 1.6 | 1.28-2 | 2.7E−05 |
| OSMR | 161 | 1.43 | 1.21-1.69 | 3.3E−05 |
| ATP13A3 | 162 | 0.69 | 0.57-0.82 | 3.3E−05 |
| GTPBP4 | 163 | 1.44 | 1.21-1.72 | 3.3E−08 |
| APOC1 | 164 | 1.44 | 1.21-1.72 | 3.7E−05 |
| MPI | 165 | 1.48 | 1.23-1.78 | 3.8E−05 |
| AHR | 166 | 1.48 | 1.28-1.79 | 3.9E−05 |
| TCERG1 | 167 | 1.57 | 1.26-1.96 | 4.6E−05 |
| CPA2 | 168 | 1.46 | 1.22-1.75 | 4.7E−05 |
| RCAN2 | 169 | 1.54 | 1.25-1.91 | 4.8E−05 |
| STEAP1 | 170 | 0.69 | 0.58-0.83 | 6.1E−05 |
| SLC39A6 | 171 | 0.71 | 0.6-0.84 | 6.1E−05 |
| IMPAD1 | 172 | 0.71 | 0.6-0.84 | 7.2E−05 |
| SLC25A32 | 173 | 0.68 | 0.56-0.82 | 7.5E−05 |
| POLD1 | 174 | 1.42 | 1.19-1.69 | 7.6E−05 |
| SPARC | 175 | 1.42 | 1.19-1.69 | 8.0E−05 |
| STAT2 | 176 | 1.41 | 1.19-1.67 | 8.8E−05 |
| NOTCH1 | 177 | 1.55 | 1.24-1.94 | 8.9E−05 |
| CCT6A | 178 | 0.66 | 0.53-0.81 | 9.3E−05 |
| ZNF146 | 179 | 0.69 | 0.57-0.83 | 9.4E−05 |
| ALDH6A1 | 180 | 1.43 | 1.19-1.71 | 9.8E−05 |
| HPGD | 181 | 0.65 | 0.53-0.79 | 2.6E−05 |
| ID3 | 182 | 1.43 | 1.19-1.72 | 0.00013 |
| SOX9 | 183 | 1.4 | 1.18-1.67 | 0.00015 |
| CDK9 | 184 | 1.38 | 1.17-1.64 | 0.00019 |
| EEF1A2 | 185 | 1.41 | 1.18-1.69 | 0.00020 |
| HEATR1 | 186 | 0.67 | 0.54-0.83 | 0.00022 |
| NCBP2 | 187 | 0.73 | 0.61-0.86 | 0.00024 |
| PMVK | 188 | 0.72 | 0.61-0.86 | 0.00027 |
| GMPS | 189 | 0.68 | 0.55-0.84 | 0.00027 |
| MAL | 190 | 1.41 | 1.17-1.69 | 0.00028 |
| KLF4 | 191 | 0.72 | 0.6-0.86 | 0.00032 |
| CDK6 | 192 | 1.34 | 1.14-1.58 | 0.00034 |
| LBR | 193 | 1.23 | 1.1-1.38 | 0.00035 |
| NUP107 | 194 | 0.68 | 0.55-0.84 | 0.00039 |
| LOX | 195 | 1.4 | 1.16-1.68 | 0.00039 |
| SCNN1B | 196 | 1.37 | 1.15-1.64 | 0.00041 |
| BCAR3 | 197 | 0.74 | 0.62-0.87 | 0.00042 |
| MMP14 | 198 | 0.73 | 0.61-0.87 | 0.00043 |
| ADAT1 | 199 | 1.39 | 1.16-1.68 | 0.00044 |
| SLAMF8 | 200 | 1.42 | 1.17-1.74 | 0.00046 |
| PRC1 | 201 | 0.71 | 0.58-0.86 | 0.00048 |
| MDFI | 202 | 1.35 | 1.13-1.6 | 0.00067 |
| SUPT16H | 203 | 1.48 | 1.18-1.85 | 0.00072 |
| PN01 | 204 | 0.74 | 0.62-0.88 | 0.00075 |
| MMP9 | 205 | 0.74 | 0.62-0.88 | 0.00079 |
| ADH1C | 206 | 0.72 | 0.6-0.88 | 0.00085 |
| SDS | 207 | 1.34 | 1.13-1.6 | 0.00090 |
| NOP56 | 208 | 0.73 | 0.61-0.88 | 0.00095 |
| SGSM3 | 209 | 1.37 | 1.13-1.65 | 0.0011 |
| FERMT1 | 210 | 0.75 | 0.64-0.89 | 0.0011 |
| PMMI | 211 | 1.41 | 1.14-1.78 | 0.0012 |
| CAPN9 | 212 | 1.4 | 1.14-1.71 | 0.0014 |
| COL6A3 | 213 | 1.32 | 1.11-1.58 | 0.0018 |
| ALDH2 | 214 | 0.75 | 0.63-0.9 | 0.0019 |
| EIF2AK2 | 215 | 0.76 | 0.64-0.9 | 0.0019 |
| SLC20A1 | 216 | 1.31 | 1.1-1.55 | 0.0021 |
| ANG | 217 | 0.74 | 0.61-0.9 | 0.0022 |
| CCT2 | 218 | 0.76 | 0.63-0.91 | 0.0023 |
| PAK1IP1 | 219 | 0.73 | 0.59-0.89 | 0.0026 |
| NID2 | 220 | 1.35 | 1.11-1.65 | 0.0027 |
| BTD | 221 | 1.39 | 1.12-1.73 | 0.0028 |
| MAOA | 222 | 0.75 | 0.62-0.91 | 0.0037 |
| XAF1 | 223 | 0.77 | 0.65-0.92 | 0.0037 |
| GPT | 224 | 0.76 | 0.63-0.92 | 0.0038 |
| COL5A2 | 225 | 1.35 | 1.1-1.66 | 0.0041 |
| PDP1 | 226 | 1.29 | 1.08-1.54 | 0.0044 |
| NOL8 | 227 | 0.77 | 0.64-0.92 | 0.0048 |
| UTP6 | 228 | 0.77 | 0.65-0.93 | 0.0048 |
| EPHB4 | 229 | 1.34 | 1.09-1.64 | 0.0051 |
| RCN1 | 230 | 0.74 | 0.6-0.92 | 0.0054 |
| CHGA | 231 | 1.27 | 1.07-1.51 | 0.0068 |
| MCM2 | 232 | 1.27 | 1.06-1.53 | 0.0087 |
| CDK12 | 233 | 0.73 | 0.58-0.93 | 0.01 |
| DPYSL2 | 234 | 1.25 | 1.05-1.49 | 0.01 |
| CPXM1 | 235 | 0.81 | 0.69-0.95 | 0.011 |
| CHSY1 | 236 | 1.27 | 1.06-1.54 | 0.011 |
| ACOX3 | 237 | 0.8 | 0.67-0.95 | 0.011 |
| GCNT4 | 238 | 1.29 | 1.05-1.58 | 0.014 |
| INTS8 | 239 | 0.77 | 0.63-0.95 | 0.014 |
| IFITM1 | 240 | 0.78 | 0.63-0.96 | 0.016 |
| ITGA2 | 241 | 1.26 | 1.04-1.51 | 0.016 |
| NCL | 242 | 0.79 | 0.65-0.96 | 0.02 |
| TOPBP1 | 243 | 0.82 | 0.69-0.97 | 0.022 |
| PRR7 | 244 | 1.21 | 1.03-1.44 | 0.024 |
| CLDN4 | 245 | 0.8 | 0.65-0.97 | 0.024 |
| TNFRSF12A | 246 | 0.82 | 0.69-0.98 | 0.024 |
| SELENBP1 | 247 | 1.25 | 1.02-1.53 | 0.033 |
| CLDN1 | 248 | 1.19 | 1.01-1.41 | 0.042 |
| SST | 249 | 0.83 | 0.69-1 | 0.046 |
| FCGBP | 250 | 0.84 | 0.71-1 | 0.052 |
| AKR7A3 | 251 | 0.83 | 0.69-1 | 0.052 |
| OAS2 | 252 | 1.19 | 0.99-1.42 | 0.057 |
| TREM2 | 253 | 1.2 | 0.99-1.45 | 0.059 |
| MEST | 254 | 1.2 | 0.99-1.46 | 0.061 |
| FPR3 | 255 | 0.86 | 0.72-1.02 | 0.076 |
| SLC25A4 | 256 | 0.86 | 0.72-1.02 | 0.079 |
| AKR1B10 | 257 | 0.86 | 0.73-1.02 | 0.082 |
| DPT | 258 | 1.16 | 0.98-1.38 | 0.09 |
| HSPH1 | 259 | 1.17 | 0.97-1.42 | 0.097 |
| DRAM1 | 260 | 0.91 | 0.81-1.02 | 0.11 |
| SCGB2A1 | 261 | 0.86 | 0.7-1.05 | 0.13 |
| F2R | 262 | 0.88 | 0.74-1.04 | 0.13 |
| GPRC5A | 263 | 0.88 | 0.74-1.04 | 0.14 |
| VSIG2 | 264 | 0.85 | 0.67-1.07 | 0.16 |
| IFIT3 | 265 | 1.14 | 0.95-1.36 | 0.16 |
| GKN1 | 266 | 1.14 | 0.94-1.39 | 0.18 |
| RBM28 | 267 | 0.9 | 0.76-1.06 | 0.22 |
| GIF | 268 | 1.11 | 0.93-1.33 | 0.23 |
| CDH3 | 269 | 1.12 | 0.92-1.35 | 0.26 |
| LIPF | 270 | 1.12 | 0.91-1.37 | 0.27 |
| MCM3 | 271 | 0.91 | 0.76-1.09 | 0.29 |
| ORC2 | 272 | 1.11 | 0.92-1.34 | 0.29 |
| PSCA | 273 | 1.1 | 0.91-1.33 | 0.31 |
| COLIBA1 | 274 | 0.95 | 0.84-1.06 | 0.34 |
| CDH11 | 275 | 1.07 | 0.91-1.26 | 0.41 |
| PSMD3 | 276 | 1.08 | 0.9-1.3 | 0.41 |

TABLE 2
Canonical discriminant function coefficients

| Gene Name | Gene ID | Coefficient |
|---|---|---|
| TUBB | 203068 | 3.061 |
| GABBR1 | 2550 | −2.006 |
| KAT2A | 2648 | .620 |
| FHOD1 | 29109 | −2.519 |
| CBFB | 865 | 2.351 |
| TNFAIP2 | 7127 | −2.227 |
| SMS | 6611 | −1.355 |
| CAPRIN1 | 4076 | .851 |
| PTGS1 | 5742 | .737 |
| ECHDC2 | 55268 | −.908 |
| GART | 2618 | −2.340 |
| FGFR4 | 2264 | .853 |
| BAZ1A | 11177 | 1.271 |
| CXCL10 | 3627 | 3.844 |
| ETFDH | 2110 | 2.345 |
| SLC12A9 | 56996 | −2.092 |
| CEP55 | 55165 | −2.782 |
| LRRC41 | 10489 | 2.541 |
| PGRMC2 | 10424 | −1.276 |
| TGS1 | 96764 | 1.374 |
| CXCL1 | 2919 | .612 |
| LIMK1 | 3984 | 1.628 |
| LAMC2 | 3918 | −2.081 |
| ENC1 | 8507 | .431 |
| ADNP | 23394 | −1.053 |
| ABCE1 | 6059 | 1.036 |
| CHORDC1 | 26973 | −1.949 |
| P4HA1 | 5033 | −.871 |
| APOE | 348 | −2.007 |
| INHBA | 3624 | −.518 |
| OSMR | 9180 | .573 |
| ATP13A3 | 79572 | .884 |
| APOC1 | 341 | .982 |
| TCERG1 | 10915 | 3.891 |
| CCT6A | 908 | −1.022 |
| ALDB6A1 | 4329 | −.511 |
| KLF4 | 9314 | −1.320 |
| SCNN1B | 6338 | −.501 |
| BCAR3 | 8412 | 2.330 |
| MMP14 | 4323 | .960 |
| PRC1 | 9055 | 1.853 |
| PNO1 | 56902 | 2.542 |
| ADH1C | 126 | .765 |
| COL6A3 | 1293 | −.698 |
| SLC20A1 | 6574 | 3.171 |
| CCT2 | 10576 | 3.061 |
| PDP1 | 54704 | −2.006 |
| NOL8 | 55035 | .620 |
| EPHB4 | 2050 | −2.519 |
| MCM2 | 4171 | 2.351 |
| CPXM1 | 56265 | −2.227 |
| NCL | 4691 | −1.355 |
| PRR7 | 80758 | .851 |

1. Use of a panel of 53 gastric cancer (GC)-related genes in preparing a medicament or system for diagnosis and prediction of metastasis, staging, and recurrence of human GC, wherein the GC related genes are (1) cell cycle related genes: CEP55, MCM2, PRC1, SCNN1B, TUBB; (2) acetylation related genes: ADNP, ABCE1, CBFB, CHORDC1, CCT6A, GART, SMS; (3) RNA/ncRNA process related genes: NOL8, NCL, PNO1; (4) extracellular matrix related genes: APOE, APOC1, CXCL10, COL6A3, CPXM1, GABBR1, INHBA, LAMC2, MMP14, TNFAIP2; (5) other genes: ADH1C, ALDH6A1, ATP13A3, BAZ1A, BCAR3, CAPRIN1, CXCL1, CCT2, ECHD2, ETFDH, ENC1, EPHB4, FHOD1, FGFR4, KAT2A, KLF4, LRRC41, LIMK1, OSMA, PTGS1, PGRMC2, P4HA1, PDP1, PRR7, SCC12A9, SLC20A1, TGS1, TCERG1; and (6) control genes: ACTB and GAPDH.

2. Use of a panel of gene probes or primers in preparing a medicament or system for diagnosis and prediction of metastasis, staging, and recurrence of human GC, wherein 53 GC related genes against which the gene probes or primers are directed are defined in claim 1.

3. The use according to claim 1, wherein the system is used to determine mRNA expression levels of 53 target genes by real-time, fluorescence-based quantitative PCR, gene chip, next-generation high-throughput sequencing, Panomics, or Nanostring technology.

4. A kit for measuring expression levels of target genes in GC, comprising the probes or primers of claim 2.
